(19) United States
(12) Patent Application Publication
(10) Pub. No.: US 2013/0225522 A1
(43) Pub. Date: Aug. 29, 2013

(54) COMPOSITION FOR RELIEVING STIMULUS OF SKIN CAUSED BY DYE

(71) Applicants: Jun Seong Park, Suwon-si (KR); Kyoung Mi Jung, Yongin-si (KR); Kyung Mi Joo, Hwaseong-si (KR); Wang Gi Kim, Suwon-si (KR); Duck Hee Kim, Seoul (KR); Han Kon Kim, Suwon-si (KR)

(72) Inventors: Jun Seong Park, Suwon-si (KR); Kyoung Mi Jung, Yongin-si (KR); Kyung Mi Joo, Hwaseong-si (KR); Wang Gi Kim, Suwon-si (KR); Duck Hee Kim, Seoul (KR); Han Kon Kim, Suwon-si (KR)

(73) Assignee: Amorepacific Corporation, Seoul (KR)

(21) Appl. No.: 13/510,763

(22) PCT Filed: Nov. 18, 2010
(86) PCT No.: PCT/KR10/08158

§ 371 Date: May 18, 2012

(30) Foreign Application Priority Data
Nov. 19, 2009 (KR) 10-2009-0112178

(51) Int. Cl. A61K 8/60; A61Q 19/00; A61Q 5/10

(52) U.S. Cl. CPC A61K 8/60; A61Q 5/10; A61Q 19/00
USPC 514/53; 514/23; 514/54

(57) ABSTRACT

The present invention relates to a composition containing a carbohydrate or a derivative thereof as an active ingredient. The composition relieves stimulus of the skin caused by dye and prevents inflammation caused by dye. In addition, the present invention relates to a hair dye composition comprising a composition containing a carbohydrate or a derivative thereof as an active ingredient. The hair dye composition reduces stimulus of the skin and inflammation caused by dye.

### TECHNICAL FIELD

This disclosure relates to a composition effective for relieving skin irritation caused by a dye.

### BACKGROUND ART

Recently, many people irrespective of age and sex have used a hair dye to cover prematurely gray hair and poliosis and to satisfy desires for expression of individuality. Such hair dyes may be classified, depending on duration time, into the following types.

First, a temporary color is intended to realize a color momentarily, and leaves no color on hair after washing with water. Such temporary dyes are nearly neutral and include color spray, color mousse, color gel, mascara or the like.

Next, a semi-permanent color is an all-in-one hair dye. It is further classified into an alkaline color and an acidic color. Although an acidic color is to be mixed with hydrogen peroxide like a permanent color, it requires a relatively low concentration of peroxide, and thus impairs hair to a lower degree than a permanent color. An alkaline color requires no mixing with peroxide, and thus causes no damage upon hair cuticles and cortex. Moreover, an alkaline color forms a glossy protective film, thereby functioning to interrupt UV rays and to protect hair. Such semi-permanent colors have a duration time of approximately 2 weeks-4 weeks. They are capable of dyeing not through chemical bonding but through ionic bonding, and include hair manicure.

Finally, a permanent color consists of a first agent (dye) and a second agent (oxidant) and is capable of dyeing through the reaction between the first agent and the second agent when they are combined with each other. Such permanent colors may be further classified into vegetable colors, fast colors and oxidative colors. Currently, oxidative colors have been used most frequently.

In general, oxidative colors contain an oxidative dye and an alkaline agent. As the oxidative dye, a dye such as m-aminophenol, p-phenylene diamine or p-aminophenol is used. Such an oxidative dye infiltrates into hair and functions to change the hair color. The alkaline agent functions to swell hair so that the oxidative dye infiltrates thereto. Typical examples of the alkaline agent include monoethanolamine and aqueous ammonia. Typical examples of the second agent include hydrogen peroxide.

### DISCLOSURE

### Technical Problem

It has now been found that a composition comprising a carbohydrate or derivative thereof as an active ingredient is effective for relieving skin irritation caused by a dye and for preventing or inhibiting inflammation caused by a dye. This disclosure is directed to providing a composition for relieving skin irritation caused by a dye and for preventing or inhibiting inflammation caused by a dye, wherein the composition comprises a carbohydrate or derivative thereof as an active ingredient.

### Technical Solution

In one general aspect, there is provided a composition for relieving skin irritation caused by a dye, comprising a carbohydrate or derivative thereof as an active ingredient.

In another general aspect, there is provided a composition for preventing or inhibiting inflammation caused by a dye, comprising a carbohydrate or derivative thereof as an active ingredient.

In still another general aspect, there is provided a hair dye composition comprising the above-mentioned composition.

### Advantageous Effects

The composition comprising a carbohydrate or derivative thereof as an active ingredient as disclosed herein is effective for relieving skin irritation caused by a dye.

The composition comprising a carbohydrate or derivative thereof as an active ingredient as disclosed herein is effective for preventing or inhibiting inflammation caused by a dye.

The hair dye composition comprising the composition comprising a carbohydrate or derivative thereof as an active ingredient as disclosed herein is effective for preventing or inhibiting skin irritation and inflammation caused by a dye.

### BEST MODE

As used herein, the term ‘skin’ means the tissue covering the body surface of an animal. In the broadest meaning of the term ‘skin’, it covers scalp and hair as well as the tissue covering the body surface such as face or body.

Hereinafter, the embodiments of the present disclosure will be described in detail.

Currently used hair dye products contain a diamine-based organic dye, surfactant, fragrance, ammonia solution, hydrogen peroxide solution, ammonium thioglycolate, fatty acid alcohol, or the like. However, among the ingredients, the diamine-based organic dye causes skin irritation. In addition, when the diamine-based organic dye is bound to skin cells and functions as an antigen, side effects, such as flare, rash, itching, urticaria, inflammation, blistering or contact dermatitis occur on the skin. Therefore, when people with sensitive skin use products containing a diamine-based organic dye, they may suffer from contact allergic dermatitis, or the like. When a hair dye is applied onto scalp sensitized due to frequent permanent, dyeing and stress, such a diamine-based organic dye is absorbed to the scalp in an excessive amount so that the user may suffer from severe irritation. In some instances, the user may not accept a dyeing procedure. Currently, irritation-relieving agents are used in combination with a hair dye to carry out a hair dyeing procedure. However, it is difficult for most irritation-relieving agents used in a dyeing procedure to provide a desired skin irritation-relieving effect due to a low pH of a hair dye product itself and oxidation process included in a dyeing procedure. In addition, when an excessive amount of irritation-relieving agent is combined with a hair dye product, it may adversely affect the dyeing process itself. Therefore, such irritation-relieving agents are limited in their applications.

In one aspect, there is provided a composition for relieving skin irritation caused by a dye and for preventing or inhibiting inflammation caused by a dye, wherein the composition comprises a carbohydrate or derivative thereof as an active ingredient.

The carbohydrate or derivative thereof generally means a compound containing carbon, hydrogen and oxygen atoms, having a formula of Cₙ(H₂O)ₙ, and containing one or more alcohol groups (—OH) and one aldehyde (—CHO) or ketone (═CO) group per molecule. However, the carbohydrate or derivative thereof also includes a compound having oxygen atoms in a number smaller than the formula by 1 (for example, deoxyribose), a compound further containing nitrogen atoms (for example, diaminosaccharide), a compound further containing sulfur atoms (for example, condroitin sulfate), or the like.

According to an embodiment, the carbohydrate or derivative thereof may be at least one selected from the group consisting of monosaccharides, oligosaccharides and polysaccharides.

Particularly, the term ‘monosaccharide’ means a unit of carbohydrate and includes a saccharide produced by acidic or enzymatic hydrolysis of polysaccharides (for example, starch or cellulose). More particularly, according to an embodiment, the monosaccharide may be rhamnose, glucose, galactose, fructose or xylose.

Rhamnose has a formula of C₆H₁₂O₅. Naturally occurring rhamnose is mainly L-type rhamnose, also called 6-deoxymannose.

Glucose has a formula of C₆H₁₂O₆ and is a typical hexose monosaccharide having an aldehyde group. Glucose may be represented by the following Chemical Formula 1. According to an embodiment, n in Chemical Formula 1 may equal to 1.

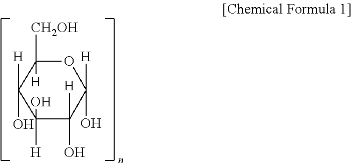

Galactose is one of hexose monosaccharides having an aldehyde group and is a type of sugar less sweet than glucose. Galactose has a formula of C₆H₁₂O₆ and may be represented by the following Chemical Formula 2. According to an embodiment, n in Chemical Formula 2 may equal to 1.

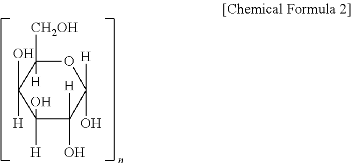

Fructose is not only a hexose but also a ketose, has a formula of C₆H₁₂O₆ and may be represented by the following Chemical Formula 3. According to an embodiment, n in Chemical Formula 3 may equal to 1.

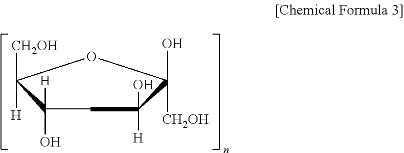

Xylose is a pentose having an aldehyde group, belongs to a group of aldopentoses and has a formula of C₅H₁₀O₅.

The term ‘oligosaccharide’, also called oligomeric saccharide, is a saccharide formed by glycoside bonding of monosaccharides. It is a generic term covering from disaccharide formed of two monosaccharides to decasaccharide formed of ten monosaccharides. The saccharide components in glycoproteins and glycolipids mostly belong to oligosaccharides. More particularly, according to an embodiment, the oligosaccharide may be lactose, sucrose or maltose. According to another embodiment, the oligosaccharide may include raffinose as a trisacchride, or stachyose as a tetrasaccharide.

Lactose is also called milk sugar and is a disaccharide formed of galactose and glucose. Lactose has a formula of C₁₂H₂₂O₁₁.

Sucrose is also called table sugar and is a disaccharide formed of glucose and fructose.

Maltose is also called malt sugar, is a disaccharide formed of two glucose molecules, and has a formula of C₁₂H₂₂O₁₁.

The term ‘polysaccharide’ includes all saccharides formed as macromolecules via glycoside bonding of two or more monosaccharides. Polysaccharides may have a molecular weight ranging from several thousands to 1,000,000 or more. According to an embodiment, polysaccharides include the compounds represented by Chemical Formulae 1 to 3, wherein n is 2 to 100,000.

According to an embodiment, polysaccharides may include homopolysaccharides having a single type of saccharides and heteropolysaccharides having at least two types of saccharides. Particularly, polysaccharides include starch, glycogen, cellulose, hemicelluloses, or pectin. More particularly, polysaccharides include dextran or starch as a glucose polymer, xylan as a xylose polymer, inulin or levan as a fructose polymer, or galactan as a galactose polymer.

The composition comprising a carbohydrate or derivative thereof as an active ingredient according to an embodiment is effective for relieving skin irritation caused by a dye and for preventing or inhibiting inflammation caused by a dye. According to another embodiment, the dye may be a diamine-based organic dye.

In another aspect, there is provided a hair dye composition comprising the composition comprising a carbohydrate or derivative thereof as an active ingredient. Since the composition comprising a carbohydrate or derivative thereof as an active ingredient is effective for relieving skin irritation caused by a dye and for preventing or inhibiting inflammation caused by a dye, the hair dye composition is also effective for alleviating skin irritation and inflammation as compared to the existing hair dye composition. More particularly, the dye may be a diamine-based organic dye.

According to an embodiment, the hair dye composition may include a first agent and a second agent. In the case of the first agent, it may be formulated into a powder, liquid, cream, lotion or gel type formulation but is not limited thereto. According to another embodiment, the hair dye composition may be an all-in-one hair dye composition in which the first agent and the second agent are integrated into a single composition. The all-in-one hair dye composition may be formulated into a powder, liquid, cream, lotion or gel type formulation but is not limited thereto.

According to an embodiment, the carbohydrate or derivative thereof may be present in the composition comprising a carbohydrate or derivative thereof as an active ingredient, in an amount of 0.1 to 80 wt % based on the total weight of the composition. According to another embodiment, the carbohydrate or derivative thereof may be present in an amount of 0.1 to 50 wt % based on the total weight of the composition. According to still another embodiment, the carbohydrate or derivative thereof may be present in an amount of 0.1 to 10 wt % based on the total weight of the composition. When the carbohydrate or derivative thereof is present in an amount less than 0.1 wt %, it is not possible to provide a sufficient effect. When the carbohydrate or derivative thereof is present in an amount greater than 80 wt %, the composition may be problematic in terms of stability.

According to an embodiment, the composition comprising a carbohydrate or derivative thereof as an active ingredient may be present in the hair dye composition comprising the composition comprising a carbohydrate or derivative thereof as an active ingredient, in an amount of 0.1 to 10 wt % based on the total weight of the hair dye composition. According to an embodiment, the composition comprising a carbohydrate or derivative thereof as an active ingredient may be present in the hair dye composition in an amount of 0.1 to 5 wt % based on the total weight of the hair dye composition. According to another embodiment, the composition comprising a carbohydrate or derivative thereof as an active ingredient may be present in the hair dye composition in an amount of 0.1 to 1 wt % based on the total weight of the hair dye composition. When the composition comprising a carbohydrate or derivative thereof as an active ingredient is present in an amount less than 0.1 wt %, it is not possible to provide a sufficient effect. When the composition comprising carbohydrate or derivative thereof as an active ingredient is present in an amount more than 80 wt %, the composition may be problematic in terms of stability.

### MODE FOR INVENTION

The examples and test examples will now be described. The following examples and test examples are for illustrative purposes only and not intended to limit the scope of this disclosure.

### Test Example 1

### Evaluation of Effect of Relieving Skin Irritation Caused by Dye and for Preventing or Inhibiting Inflammation Caused by Dye

To evaluate an effect of relieving skin irritation caused by a dye and for preventing or inhibiting inflammation caused by a dye, the mouse ear swelling test (MEST) using a mouse is carried out in the manner as described below.

A 7 weeks-aged mouse subject (BALC/c) is obtained and subjected to quarantine and adaptation for a period of 10 days. Then, the mouse is dehaired at its abdominal portion, and a main ingredient of dye, i.e. 3% paraphenylene diamine (PPD) is applied thereto in combination with a monosaccharide (glucose, galactose or fructose) or disaccharide (maltose, lactose or sucrose) for 7 days three times a day. As a control, each of dimethyl sulfoxide (DMSO) solvent and 3% PPD is applied. Twenty-four hours after the final application, the mouse ear is weighed. After weighing, the mouse ear is fixed in formalin and subjected to hematoxylin & eosin (H&E) staining for a histopathological test. Then, the thickness of the mouse ear is determined. Herein, the weight of the mouse ear is measured by biopsy of the ear of the mouse subject (Balb/c), to which each test sample is applied, using a 6 mm punch. The thickness of the mouse ear is determined by measuring the thickness of the ear of the mouse subject (Balb/c) stained after weighing the mouse ear weight by using a microscope. The results are shown in the following Table 1.

TABLE 1

| Sample | Ear weight (mg) | Ear thickness (μm) |
|---|---|---|
| Control (DMSO) | 8.14 ± 1.11 | 237.34 ± 19.24 |
| PPD (3%) | 14.74 ± 0.23 | 433.28 ± 11.57 |
| PPD + glucose | 9.11 ± 1.20 | 241.22 ± 41.54 |
| PPD + galactose | 9.09 ± 1.03 | 243.93 ± 37.48 |
| PPD + fructose | 8.64 ± 1.15 | 248.43 ± 25.57 |
| PPD + maltose | 9.35 ± 1.32 | 259.28 ± 21.74 |
| PPD + lactose | 8.38 ± 1.11 | 259.52 ± 46.04 |
| PPD + sucrose | 8.22 ± 1.41 | 243.54 ± 40.01 |

It can be seen from the above results that application of paraphenylene diamine in combination with a carbohydrate leads to a significant drop in ear weight and thickness as compared to application of paraphenylene diamine alone. In other words, this suggests that a carbohydrate or derivative thereof alleviates skin irritation, inflammation and edema caused by paraphenylene diamine. Therefore, it can be seen that a carbohydrate or derivative thereof is effective for relieving skin irritation caused by a diamine-based organic dye and for preventing or inhibiting inflammation caused by a diamine-based organic dye. Different carbohydrates or derivatives thereof may be used in combination.

Hereinafter, some formulation examples for the hair dye composition comprising the composition comprising a carbohydrate or derivative thereof as an active ingredient will be provided. However, the hair dye composition may be formulated into various formulation types in addition to the following formulation examples, which are for illustrative purposes only and not intended to limit the scope of this disclosure.

### Formulation Example 1

### Powder Type First Agent Hair Dye Composition

| Ingredients | Wt % |
|---|---|
| Fructose | 60.0 |

| | | |
|---|---|---|
| Dye | Paraphenylene diamine | 10.0 |
| Thickener | Starch | 10.0 |
| pH modifier | L-arginine | 10.0 |
| Reducing agent | Sodium pyrosulfite | 10.0 |

### Formulation Example 2

### Gel Type First Agent Hair Dye Composition

| Ingredients | Wt % |
|---|---|
| Glucose | 1.0 |
| Paraphenylene diamine | 3.0 |
| Hydroxyethyl cellulose | 0.5 |
| L-arginine | 2.0 |
| Sodium pyrosulfite | 1.0 |
| Purified water | To 100 |

### Formulation Example 3

### All-in-One Hair Dye Composition

| Ingredients | Wt % |
|---|---|
| Sucrose | 1.0 |

| | | |
|---|---|---|
| Dye | Paraphenylene diamine | 10.0 |
| Thickener | Starch | 10.0 |
| pH modifier | L-arginine | 10.0 |
| Reducing agent | Sodium pyrosulfite | 10.0 |
| Water-soluble iron salt | Ferrous sulfate | 2.0 |
| | heptahydrate | |
| Purified water | Purified water | To 100 |

1-8. (canceled)

9. A method for relieving skin irritation caused by a dye of a subject, comprising:
administering to the subject an effective amount of a carbohydrate or derivative thereof that relieves skin irritation caused by a dye of the subject.

10. The method according to claim 9, wherein the carbohydrate or derivative thereof is at least one selected from the group consisting of monosaccharides, oligosaccharides and polysaccharides.

11. The method according to claim 10, wherein the monosaccharides comprise rhamnose, glucose, galactose, fructose or xylose.

12. The method according to claim 10, wherein the oligosaccharides comprise lactose, sucrose or maltose.

13. The method according to claim 10, wherein the polysaccharides comprise dextran or starch which are a glucose polymer, xylan which is a xylose polymer, inulin or levan which are a fructose polymer, or galactan which is a galactose polymer.

14. The method according to claim 9, wherein the dye is a diamine-based organic dye.

15. The method according to claim 9, wherein the carbohydrate or derivative thereof is administered in a form of a hair dye composition comprising the carbohydrate or derivative thereof.

16. A method for preventing or inhibiting skin inflammation caused by a dye of a subject, comprising:
administering to the subject an effective amount of a carbohydrate or derivative thereof that prevents or inhibits skin inflammation caused by a dye of the subject.

17. The method according to claim 16, wherein the carbohydrate or derivative thereof is at least one selected from the group consisting of monosaccharides, oligosaccharides and polysaccharides.

18. The method according to claim 17, wherein the monosaccharides comprise rhamnose, glucose, galactose, fructose or xylose.

19. The method according to claim 17, wherein the oligosaccharides comprise lactose, sucrose or maltose.

20. The method according to claim 17, wherein the polysaccharides comprise dextran or starch which are a glucose polymer, xylan which is a xylose polymer, inulin or levan which are a fructose polymer, or galactan which is a galactose polymer.

21. The method according to claim 16, wherein the dye is a diamine-based organic dye.

22. The method according to claim 16, wherein the carbohydrate or derivative thereof is administered in a form of a hair dye composition comprising the carbohydrate or derivative thereof.
